(19) United States
(12) Patent Application Publication
(10) Pub. No.: US 2018/0311141 A1
(43) Pub. Date: Nov. 1, 2018

(54) ANHYDROUS SCREENING COMPOSITION COMPRISING A SILICONE FATTY PHASE

(71) Applicant: L'OREAL, Paris (FR)

(72) Inventors: Florence L'ALLORET, Chevilly La Rue (FR); Marina GROC-BIDAULT, Chevilly La Rue (FR); Pascal ARNAUD, Chevilly La Rue (FR)

(21) Appl. No.: 15/756,288

(22) PCT Filed: Sep. 7, 2016
(86) PCT No.: PCT/EP2016/071103

§ 371 Date: Feb. 28, 2018

(30) Foreign Application Priority Data
Sep. 10, 2015 (FR) 1558396

(51) Int. Cl. A61K 8/893; A61K 8/34; A61K 8/35; A61K 8/37; A61K 8/49; A61K 8/58; A61Q 17/04; A61Q 19/08

(52) U.S. Cl. CPC A61K 8/893; A61K 8/34; A61K 8/35; A61K 8/37; A61K 2800/262; A61K 8/585; A61Q 17/04; A61Q 19/08; A61K 2800/31; A61K 8/4966

(57) ABSTRACT

The present invention relates to a fluid anhydrous composition comprising, especially in a cosmetically acceptable medium: a) at least 20% of a silicone fatty phase, and b) at least 5% of one or more lipophilic organic UV screening agents, and c) at least one or more C₂-C₈ monoalcohols. The present invention also relates to the use of said composition for the manufacture of products for the cosmetic treatment of the skin, the nails, the hair, the eyelashes, eyebrows and/or the scalp, especially care products, anti-sun products, makeup products and perfuming products.

The present invention relates to an anhydrous composition comprising, especially in a cosmetically acceptable medium:

a) at least 20% gloss of one or more silicone oils, and
b) at least 5% of one or more lipophilic organic UV screening agents, and
c) at least one or more C₂-C₈ monoalcohols.

It is known that light radiation with wavelengths of between 280 nm and 400 nm makes possible browning of the human epidermis and that rays with wavelengths more particularly of between 280 and 320 nm, known under the name of UV-B, cause erythemas and skin burns which may be harmful to the development of natural tanning. For these reasons, as well as for aesthetic reasons, there is a constant demand for means for controlling this natural tanning in order thus to control the colour of the skin; this UV-B radiation should thus be screened out.

It is also known that UV-A rays, with wavelengths between 320 and 400 nm, which cause tanning of the skin, are liable to induce adverse changes therein, in particular in the case of sensitive skin or skin that is continually exposed to solar radiation. UV-A rays cause in particular a loss in the elasticity of the skin and the appearance of wrinkles, resulting in premature skin ageing. They promote triggering of the erythemal reaction or amplify this reaction in certain individuals and may even be the cause of phototoxic or photoallergic reactions. Thus, for aesthetic and cosmetic reasons, such as the preservation of the natural elasticity of the skin, for example, more and more people wish to control the effect of UV-A rays on their skin. It is therefore desirable also to screen out UV-A radiation.

For the purpose of protecting the skin and keratin materials against UV radiation, anti-sun compositions comprising organic screening agents that are active in the UV-A range and in the UV-B range are generally used.

Many cosmetic compositions intended for the photoprotection (UV-A and/or UV-B) of the skin have been provided to date. Fluid formulations that are easy for the users to apply to the skin are most particularly sought.

Among the fluid anti-sun compositions proposed hitherto, anhydrous formulations of anti-sun oil type are particularly sought on account of their easy and pleasant application to the skin and their good water resistance. However, they are not particularly widespread on the anti-sun products market due to the fact that it is difficult to obtain a sun protection factor of greater than 10 and also a UVA protection factor that satisfies the ratio stipulated by the various regulations concerning anti-sun products, especially of greater than 3 and preferably greater than 5.

The sun protection factor (SPF) is expressed mathematically as the ratio of the exposure time necessary to reach the erythema-forming threshold with the UV screening agent to the time necessary to reach the erythema-forming threshold without UV screening agent. It is evaluated in vivo especially according to the international method published by Colipa/CTFA SA/JCIA (May 2006).

To characterize the protection with regard to UV-A radiation, the PPD (persistent pigment darkening) method, which measures the colour of the skin observed 2 to 4 hours after exposure of the skin to UV-A radiation, is particularly recommended and used. This method has been adopted since 1996 by the Japanese Cosmetic Industry Association (JCIA) as the official test procedure for the UV-A labelling of products and is frequently used by test laboratories in Europe and the United States (Japan Cosmetic Industry Association Technical Bulletin. Measurement Standards for UVA protection efficacy. Issued Nov. 21, 1995 and effective as of Jan. 1, 1996).

The UVA_{PPD} protection factor (UVA_{PPD} PF) is expressed mathematically by the ratio of the UV-A radiation dose necessary to reach the pigmentation threshold with the UV screening agent (MPPDp) to the UV-A radiation dose necessary to reach the pigmentation threshold without UV screening agent (MPPDnp).

$${UVA}_{PPD} PF = \frac{MPPDp}{MPPDnp}$$

Specifically, the main difficulties encountered in the manufacture of anti-sun oils for attempting to increase the sun protection factors are those of obtaining a formulation that is fluid, transparent and also stable, and that is cosmetically pleasant. Compositions are especially sought that have little or no whitening effect on the skin during spreading and a non-tacky and non-shiny finish.

Moreover, consumers are increasingly seeking cosmetic products that spread easily and rapidly on the skin in the form of a deposit which should not be thick but on the contrary should blend in as much as possible with the support.

It has already been proposed in application FR2236173 to use hydrocarbon-based oils in fluid anti-sun compositions in order to obtain high sun protection factors. These compositions do not have very good cosmetic properties.

There is therefore a need to find novel fluid anhydrous anti-sun compositions, which are transparent and stable over time, making it possible to achieve higher sun protection and UVA factors and improved cosmetic properties.

The Applicant Company has now discovered, surprisingly, that these objectives can be achieved with an anhydrous composition comprising, especially in a cosmetically acceptable medium:

a) at least 20% by weight, preferably 22% by weight, of a silicone fatty phase, relative to the total weight of the composition,
b) at least 5% of a lipophilic organic UV screening agent, and
c) at least one C₂-C₈ monoalcohol.

This discovery forms the basis of the present invention.

The present invention thus relates to an anhydrous composition comprising, especially in a cosmetically acceptable medium:

a) at least 20% by weight, preferably 22% by weight, of a silicone fatty phase, relative to the total weight of the composition,
b) at least 5% by weight, relative to the total weight of the composition, of one or more lipophilic organic UV screening agents, and
c) at least one or more C₂-C₈ monoalcohols.

The invention also relates to a cosmetic process for limiting the darkening of the skin and/or improving the colour and/or uniformity of the complexion, comprising the application, to the surface of the keratin material, of a composition as defined previously.

It also relates to a cosmetic process for preventing and/or treating the signs of ageing of a keratin material, comprising the application, to the surface of the keratin material, of a composition as defined previously.

Other characteristics, aspects and advantages of the invention will become apparent on reading the detailed description which follows.

The term “cosmetically acceptable” means compatible with the skin and/or its integuments, having a pleasant colour, odour and feel and not causing any unacceptable discomfort (stinging, tautness or redness) liable to discourage the consumer from using this composition.

The term “anhydrous composition” is intended to mean a composition containing less than 5% by weight of water relative to the total weight of the composition, or even less than 2% by weight of water, and especially free of water, the water not being added during the preparation of the composition but corresponding to the residual water provided by the mixed ingredients.

Within the meaning of the present application, the term “fatty phase” is intended to mean a phase which is fatty at room temperature (25° C.) and atmospheric pressure (760 mmHg), composed of one or more fatty substances which are compatible with one another.

Within the meaning of the present application, the term “liquid fatty phase” is intended to mean a fatty phase which is liquid at room temperature (25° C.) and atmospheric pressure (760 mmHg), composed of one or more fatty substances which are liquid at room temperature, also known as oils, which are compatible with one another.

The term “lipophilic” is intended to mean any cosmetic or dermatological compound which can be fully dissolved in molecular form in a liquid fatty phase or else which can be dissolved in colloidal form (for example in micellar form) in a liquid fatty phase.

The term “oil” is intended to mean a fatty substance that is liquid at room temperature (20 to 25° C.).

The term “hydrocarbon-based oil” is intended to mean any oil mainly comprising carbon and hydrogen atoms, and possibly ester, ether, fluoro, carboxylic acid, alcohol, silicone, amine, phenyl and/or amino acid groups.

The term “silicone oil” is intended to mean an oil comprising at least one silicon atom and especially at least one Si—O group.

The term “stable compositions” is intended to mean compositions which, after 1 month, and preferably 2 months of storage at any temperatures between 4° C. and 45° C., do not exhibit any macroscopic change in colour, in odour or in viscosity, nor any variation in microscopic appearance. For certain fluid compositions of the invention, a phenomenon of sedimentation may take place over time—simple manual shaking of these compositions makes it possible to return to the initial homogeneity.

In a preferred mode of the invention, the compositions are fluid. Within the meaning of the invention, the term “fluid composition” is intended to mean a composition that is not in solid form and whose viscosity, measured using a Rheomat 180 viscometer at 25° C. at a spin speed of 200 rpm after 30 seconds of rotation, is less than 0.5 Pa·s, more preferentially less than 0.2 Pa·s and more particularly ranging from 0.0001 Pa·s to 0.1 Pa·s.

### Silicone Fatty Phase

The compositions of the invention comprise at least 20% by weight, preferably at least 22% by weight, relative to the total weight of the composition, of a silicone fatty phase.

The silicone fatty phase preferably represents from 25% to 94% by weight and preferably from 30% to 50% by weight relative to the total weight of the composition.

The silicone fatty phase is preferably liquid.

The silicone fatty phase comprises one or more silicone oils which may be volatile or non-volatile, preferably volatile.

Within the meaning of the invention, the term “volatile oil” is intended to mean an oil that is capable of evaporating on contact with the skin or the keratin fibre in less than one hour, at room temperature and atmospheric pressure. The volatile oil(s) of the invention are volatile cosmetic oils, which are liquid at room temperature, having a nonzero vapour pressure, at room temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg), and more particularly ranging from 1.3 Pa to 1300 Pa (0.01 to 10 mmHg).

The term “non-volatile oil” is intended to mean an oil that remains on the skin or the keratin fibre at room temperature and atmospheric pressure for at least several hours, and that in particular has a vapour pressure of less than 10⁻³ mmHg (0.13 Pa).

The non-volatile silicone oils may be chosen especially from non-volatile polydimethylsiloxanes (PDMSs), polydimethylsiloxanes comprising alkyl or alkoxy groups that are pendent and/or at the end of a silicone chain, these groups each having from 2 to 24 carbon atoms, phenyl silicones, such as phenyl trimethicones, phenyl dimethicones, phenyltrimethylsiloxydiphenylsiloxanes, diphenyl dimethicones, diphenylmethyldiphenyltrisiloxanes, and 2-phenylethyltrimethyl siloxysilicates, and a mixture thereof.

Examples of volatile silicone oils that may be mentioned include volatile linear or cyclic silicone oils, in particular those with a viscosity ≤8 centistokes (8×10⁻⁶ m²/s) and in particular having from 2 to 7 silicon atoms, these silicones optionally comprising alkyl or alkoxy groups having from 1 to 10 carbon atoms. As volatile silicone oil that may be used in the invention, mention may be made especially of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Mention may also be made of the volatile linear alkyltrisiloxane oils of general formula (I):

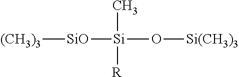

where R represents an alkyl group comprising from 2 to 4 carbon atoms, one or more hydrogen atoms of which may be substituted by a fluorine or chlorine atom.

Among the oils of general formula (I), mention may be made of:
3-butyl-1,1,1,3,5,5,5-heptamethyltrisiloxane,
3-propyl-1,1,1,3,5,5,5-heptamethyltrisiloxane, and
3-ethyl-1,1,1,3,5,5,5-heptamethyltrisiloxane,
   corresponding to the oils of formula (I) for which R is, respectively, a butyl group, a propyl group or an ethyl group.

The composition preferably comprises one or more volatile silicone oils and one or more non-volatile silicone oils.

The compositions of the invention comprise at least 20% by weight, preferably at least 22% by weight, relative to the total weight of the composition, of a silicone oil, preferably from 25% to 94% by weight and preferably from 30% to 50% by weight relative to the total weight of the composition.

The silicone fatty phase may also comprise additional silicones different from the above silicone oils, especially silicone elastomers.

### Lipophilic Organic UV Screening Agents

They may be chosen especially from para-aminobenzoic acid derivatives, salicylic derivatives, cinnamic derivatives, benzophenones and aminobenzophenones, anthranilic derivatives, dibenzoylmethane derivatives, β,β-diphenylacrylate derivatives, benzylidenecamphor derivatives, phenylbenzimidazole derivatives, benzotriazole derivatives, triazine derivatives, bis-resorcinyl triazines, imidazoline derivatives, benzalmalonate derivatives, 4,4-diarylbutadiene derivatives, benzoxazole derivatives and merocyanines, and mixtures thereof.

Among the lipophilic organic UVA screening agents that are capable of absorbing UV from 320 to 400 nm, mention may be made of

### Dibenzoylmethane Derivatives:

4-isopropyldibenzoylmethane, sold under the name Eusolex 8020 by Merck, and corresponding to the following formula:

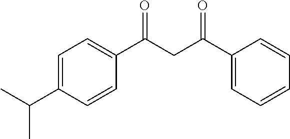 [SMILES: CC(C)c1ccc(C(=O)CC(=O)c2ccccc2)cc1]

1-(4-methoxy-1-benzofuran-5-yl)-3-phenylpropane-1,3-dione, provided for sale by Quest under the name of Pongamol, of formula:

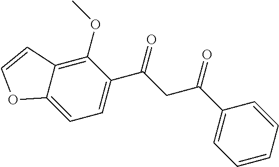 [SMILES: COc1c(C(=O)CC(=O)c2ccccc2)ccc2occc12]

1-(4-tert-butylphenyl)-3-(2-hydroxyphenyl)propane-1,3-dione of formula:

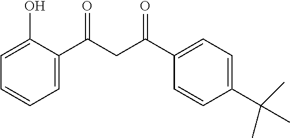 [SMILES: CC(C)(C)c1ccc(C(=O)CC(=O)c2ccccc2O)cc1]

butylmethoxydibenzoylmethane, sold especially under the trade name Parsol 1789 by Hoffmann LaRoche.

### Aminobenzophenones:

n-Hexyl 2-(4-diethylamino-2-hydroxybenzoyl)benzoate, sold under the trade name Uvinul A+.

### Anthranilic Derivatives:

Menthyl anthranilate sold under the trade name Neo Heliopan MA by Haarmann and Reimer.

### 4,4-Diarylbutadiene Derivatives:

1,1-dicarboxy(2,2′-dimethylpropyl)-4,4-diphenylbutadiene.

The preferential compounds are:

Butyl methoxy dibenzoylmethane,

n-Hexyl 2-(4-diethylamino-2-hydroxybenzoyl)benzoate.

Among the lipophilic organic UVB screening agents that are capable of absorbing UV from 280 to 320 nm, mention may be made of

para-Aminobenzoates:

Ethyl PABA,

Ethyl dihydroxypropyl PABA,

Ethylhexyl Dimethyl PABA (Escalol 507 from ISP).

### Salicylic Derivatives:

Homosalate sold under the name Eusolex HMS by Rona/EM Industries,

Ethylhexyl salicylate sold under the name Neo Heliopan OS by Haarmann & Reimer,

Dipropylene glycol salicylate sold under the name Dipsal by Scher,

TEA salicylate sold under the name Neo Heliopan TS by Haarmann & Reimer.

### Cinnamates:

Ethylhexyl methoxycinnamate sold in particular under the trade name Parsol MCX by Hoffmann LaRoche,

Isopropyl methoxycinnamate,

Isoamyl methoxycinnamate sold under the trade name Neo Heliopan E 1000 by Haarmann & Reimer,

diisopropyl methyl cinnamate,

Cinoxate,

Glyceryl Ethylhexanoate Dimethoxycinnamate.

### β,β′-Diphenylacrylate Derivatives:

Octocrylene sold especially under the trade name Uvinul N539 by BASF,

Etocrylene sold especially under the trade name Uvinul N35 by BASF.

### Benzylidenecamphor Derivatives:

3-Benzylidenecamphor manufactured under the name Mexoryl SD by Chimex,

Methylbenzylidenecamphor sold under the name Eusolex 6300 by Merck,

Polyacrylamidomethylbenzylidenecamphor manufactured under the name Mexoryl SW by Chimex.

### Triazine Derivatives:

Ethylhexyltriazone sold especially under the trade name Uvinul T150 by BASF,

2,4,6-tris(dineopentyl 4′-aminobenzalmalonate)-s-triazine,

2,4,6-tris(diisobutyl 4′-aminobenzalmalonate)-s-triazine,

2,4-bis(dineopentyl 4′-aminobenzalmalonate)-6-(n-butyl 4′-aminobenzoate)-s-triazine,

2,4-bis(n-butyl 4′-aminobenzoate)-6-(aminopropyltrisiloxane)-s-triazine.

### Imidazoline Derivatives:

Ethylhexyl dimethoxybenzylidene dioxoimidazoline propionate.

### Benzalmalonate Derivatives:

Polyorganosiloxanes containing benzalmalonate functions, for instance Polysilicone-15, sold under the trade name Parsol SLX by Hoffmann LaRoche Dineopentyl 4′-methoxybenzalmalonate.

### Merocyanine Derivatives:

Octyl 5-N,N-diethylamino-2-phenylsulfonyl-2,4-pentadienoate

The preferential compounds are:

Homosalate,

Ethylhexyl salicylate,

Ethylhexyl methoxycinnamate,

Octocrylene,

Ethylhexyl triazone,

2,4-bis(n-butyl 4′-aminobenzoate)-6-(aminopropyltrisiloxane)-s-triazine,

Octyl 5-N,N-diethylamino-2-phenylsulfonyl-2,4-pentadienoate.

Among the broad-spectrum lipophilic organic screening agents capable of absorbing UVA and UVB radiation, mention may be made of:

### Benzophenone Derivatives

Benzophenone-1 sold under the trade name Uvinul 400 by BASF,

Benzophenone-2 sold under the trade name Uvinul D50 by BASF,

Benzophenone-3 or oxybenzone sold under the trade name Uvinul M40 by BASF,

Benzophenone-5,

Benzophenone-6 sold under the trade name Helisorb 11 by Norquay,

Benzophenone-8 sold under the trade name Spectra-Sorb UV-24 by American Cyanamid,

Benzophenone-10,

Benzophenone-11,

Benzophenone-12.

### Benzotriazole Derivatives:

Drometrizole Trisiloxane, sold under the name Silatrizole by Rhodia Chimie,

Bumetrizole sold under the name Tinoguard AS by Ciba-Geigy.

Bis-resorcinyl triazine Derivatives:

Bis(ethylhexyloxyphenol)methoxyphenyltriazine sold under the trade name Tinosorb S by Ciba-Geigy.

### Benzoxazole Derivatives:

2,4-Bis[4-[5-(1,1-dimethylpropyl)benzoxazol-2-yl]phenylimino]-6-[(2-ethylhexyl)imino]-1,3,5-triazine, sold under the name of Uvasorb K2A by Sigma 3V.

The preferential compounds are:

Benzophenone-3,

Drometrizole Trisiloxane,

Bis(ethylhexyloxyphenol)methoxyphenyltriazine.

The lipophilic organic screening agents are generally present in the compositions according to the invention in proportions ranging from 5% to 50% by weight relative to the total weight of the composition, and preferably ranging from 10% to 40% by weight, more particularly from 15% to 35% by weight relative to the total weight of the composition.

C₂-C₈ Monoalcohol

The compositions of the invention comprise at least one monoalcohol comprising from 2 to 8 carbon atoms, especially from 2 to 6 carbon atoms, and in particular from 2 to 4 carbon atoms.

The compositions of the invention may comprise one or more monoalcohol(s).

This monoalcohol may be represented, for example, by the formula RₐOH, in which Rₐ represents a linear or branched alkyl group comprising from 2 to 8 carbon atoms, preferably from 2 to 6 carbon atoms, and in particular from 2 to 4 carbon atoms.

As monoalcohol, mention may be made of ethanol, isopropanol, propanol or n-butanol.

According to one embodiment, the compositions of the invention comprise ethanol.

The amount of monoalcohol(s) ranges from 1% to 30% by weight in the composition, preferably from 3% to 25% by weight and even more preferentially from 5% to 15% by weight relative to the total weight of said composition.

The amounts by weight of monoalcohol(s) correspond either to the amount by weight of the monoalcohol if the composition only comprises just one monoalcohol, or to the total amount by weight of all the monoalcohols if the composition comprises a mixture of several monoalcohols.

### Additional Oils

The compositions according to the invention may also contain one or more additional, non-screening oils which will preferably be chosen from the hydrocarbon-based oils.

The additional oils may be volatile or non-volatile and preferably volatile.

The hydrocarbon-based oil(s) are preferably present in the composition of the invention at concentrations ranging from 5% to 40% by weight and more preferentially from 10% to 30% by weight relative to the total weight of the composition, and more preferentially from 15% to 25% by weight.

Mention may especially be made, as non-volatile hydrocarbon-based oils which may be used according to the invention, of:

(i) hydrocarbon-based oils of plant origin such as triglyceride esters, which are generally fatty acid triesters of glycerol, the fatty acids of which may have chain lengths varying from C₄ to C₂₄, these chains possibly being linear or branched, and saturated or unsaturated; these oils are especially wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion-flower oil and musk rose oil; or else caprylic/capric acid triglycerides, such as those sold by Stéarineries Dubois or those sold under the names Miglyol 810, 812 and 818 by Dynamit Nobel,
(ii) synthetic ethers having from 10 to 40 carbon atoms, such as especially dialkyl ethers and in particular dicaprylyl ether;
(iii) linear or branched hydrocarbons of mineral or synthetic origin, such as petroleum jelly, polydecenes, hydrogenated polyisobutene, such as Parleam, squalane and mixtures thereof;
(iv) synthetic esters, such as, for example, the oils of formula RCOOR′ in which R represents a linear or branched fatty acid residue comprising from 1 to 40 carbon atoms and R′ represents a hydrocarbon-based chain that is especially branched, containing from 1 to 40 carbon atoms, with the proviso that R+R′≥10, for instance purcellin oil (cetostearyl octanoate), isopropyl myristate, isopropyl palmitate, C₁₂-C₁₅ alkyl benzoate, such as the product sold under the trade name Finsolv TN or Witconol TN by Witco or Tegosoft TN by Evonik Goldschmidt, 2-ethylphenyl benzoate, such as the commercial product sold under the name X-Tend 226 by ISP, isopropyl lanolate, hexyl laurate, diisopropyl adipate, isononyl isononanoate, oleyl erucate, 2-ethylhexyl palmitate, isostearyl isostearate, alcohol or polyalcohol octanoates, decanoates or ricinoleates, such as propylene glycol dioctanoate; hydroxylated esters, such as isostearyl lactate, diisostearyl malate; and pentaerythritol esters; citrates or tartrates, such as linear C₁₂-C₁₃ dialkyl tartrates, such as those sold under the name Cosmacol ETI by Enichem Augusta Industriale, and also linear C₁₄-C₁₅ dialkyl tartrates such as those sold under the name Cosmacol ETL by the same company; acetates, sebacates such as C₁-C₁₂ dialkyl sebacates, in particular diisopropyl sebacate;
(v) fatty alcohols which are liquid at ambient temperature and which have a branched and/or unsaturated carbon chain having from 12 to 26 carbon atoms, such as octyldodecanol, isostearyl alcohol, oleyl alcohol, 2-hexyldecanol, 2-butyloctanol or 2-undecylpentadecanol;
(vi) higher fatty acids, such as oleic acid, linoleic acid or linolenic acid;
(vii) C₄-C₁₂ dialkyl carbonates, such as dicaprylyl carbonate, such as the product sold under the name Cetiol CC by Cognis;
(viii) fatty amides, such as isopropyl N-lauroyl sarcosinate, such as the product sold under the trade name Eldew SL205 from Ajinomoto;
and mixtures thereof.

The volatile hydrocarbon-based oils may be chosen from hydrocarbon-based oils having from 8 to 16 carbon atoms, and especially branched C₈-C₁₆ alkanes, such as C₈-C₁₆ isoalkanes of petroleum origin (also known as isoparaffins), such as isododecane (also known as 2,2,4,4,6-pentamethylheptane), isodecane and isohexadecane, linear C₈-C₁₆ alkanes, for example the mixtures of n-undecane (C₁₁) and n-tridecane (C₁₃) sold under the reference CETIOL UT by Cognis, and for example the oils sold under the trade names Isopar or Permethyl, branched C₈-C₁₆ esters such as isohexyl neopentanoate, and mixtures thereof. Other volatile hydrocarbon-based oils, for instance petroleum distillates, especially those sold under the name Shell Solt by Shell, may also be used. According to one embodiment, the additional volatile oils are chosen from volatile hydrocarbon-based oils having from 8 to 16 carbon atoms and mixtures thereof.

Among the hydrocarbon-based oils which may be used according to the invention, preference is more particularly given to C₈-C₁₆ alkanes such as isododecane, synthetic esters and especially diisopropyl sebacate.

According to a preferred mode of the invention, the compositions may comprise one or more surfactants preferably having an HLB of less than or equal to 5. The term “HLB of less than or equal to 5” means a surfactant having, at 25° C., an HLB balance (hydrophilic-lipophilic balance), within the Griffin meaning, of less than or equal to 5. As is well known, HLB (Hydrophilic-Lipophilic Balance) is understood to mean the equilibrium between the size and the strength of the hydrophilic group and the size and the strength of the lipophilic group of the surfactant. The HLB value according to Griffin is defined in J. Soc. Cosm. Chem. 1954 (volume 5), pages 249-256.

The surfactants are preferably nonionic. Use may be made in particular of the surfactants that are cited in McCutcheon's Emulsifiers & Detergents, International edition of 1998 et seq.

The surfactants of the invention may be chosen from oil-soluble surfactants and may belong to the following families:
(C₂-C₄) oxyalkylenated polydimethylsiloxanes with 2 to 50 mol of alkylene oxide, such as polyoxyethylenated polydimethylsiloxanes with 2 to 50 mol of ethylene oxide, preferably from 5 to 20 mol of ethylene oxide,
polyoxyalkylene alkyl esters or ethers (1 to 15 mol, preferably 1 to 5 mol),
glycosylated (1 to 10 mol) or non-glycosylated polyethylene glycol (1 to 50) alkyl esters or ethers;
glyceryl or polyglyceryl (1 to 10 mol) alkyl esters or ethers;
sucrose alkyl esters or ethers;
pentaerythritol alkyl esters or ethers;
sorbitan alkyl esters or ethers;
ethoxylated derivatives of cholesterol.

The term “alkyl” is intended to mean one or more C₈ to C₃₀ branched or unbranched, saturated or unsaturated fatty chains.

Examples of such surfactants with an HLB of less than or equal to 5 are PEG-10 Dimethicone, PEG-14 Dimethicone, Cetyl PEG/PPG-10/1 Dimethicone, Steareth 2, Beheneth 2, Oleth 2, sucrose distearate, glyceryl monooleate, glyceryl stearate, glyceryl isostearate, diglyceryl distearate, tetraglyceryl tristearate, decaglyceryl decastearate, diglyceryl monostearate, hexaglyceryl tristearate, decaglyceryl pentastearate, sorbitan monostearate, sorbitan tristearate, diethylene glycol monostearate, the glycerol ester of palmitic and stearic acids, the monostearate polyoxyethylenated with 2 EO (comprising 2 oxyethylene units), glyceryl mono- and dibehenate, and pentaerythrityl tetrastearate.

According to a preferred mode of the invention, the surfactant used is PEG-10 Dimethicone.

The surfactants preferably having an HLB of less than or equal to 5 may be present in the compositions of the invention at a content ranging from 0.1% to 3% by weight relative to the total weight of the composition, preferably from 0.5% to 2.5% by weight, and even more preferably from 1% to 2%.

According to a preferred mode of the invention, the compositions may comprise particles which are insoluble in the fatty phase and which do not have UV screening properties. These particles make it possible to obtain excellent sensory properties of the compositions after application to the skin. The nature of these particles may be chosen from silicone elastomers such as the vinyl dimethicone/methicone silsesquioxane crosspolymer sold by Shin-Etsu under the name KSP100, polymethylsilsesquioxanes such as, for example, methylsilsesquioxane resin microbeads sold under the name Tospearl 145A by Momentive Performance Materials, the methylsilanol/silicate crosspolymer sold under the name NLK 506 by Takemoto Oil and Fat, nylon such as Orgasol 2002 sold by Arkema, poly(methyl methacrylate), such as the hollow PMMA spheres sold under the name Covabeads LH 85 by Sensient, talc, or hydrophobic silica. The compositions of the invention preferably comprise silicone elastomer particles, especially such as KSP 100 sold by Shin-Etsu.

By particles insoluble in the fatty phase, it means that particles having a solubility in the fatty phase of less than 0.1% (0.1 g per 100 g of fatty phase) at 25° C. and at an atmospheric pressure of 760 mmHg.

The particles which are insoluble in the compositions of the invention may be present in the compositions of the invention at a content ranging from 1% to 10% by weight relative to the total weight of the composition, preferably from 1.5% to 8% by weight, and even more preferably from 3% to 6%.

According to another preferred mode of the invention, the compositions comprise pigments such as titanium oxide or black, red or yellow iron oxide. These pigments make it possible to obtain colour effects on the skin or skin tone correcting effects.

The pigment(s) may be present in the compositions of the invention at a content ranging from 0.5% to 10% by weight relative to the total weight of the composition, preferably from 1% to 8% by weight, and even more preferably from 2% to 6%.

According to a preferred mode of the invention, the compositions comprising a fatty phase gelling agent such as a hydrophobized clay, a hydrophobized silica, a silicone elastomer, an oil gelling polymer of C₈-C₃₀ alkyl acrylate type, a silicone polyamide, an alkyl palmitate or else a block polymer of hydrogenated styrene/butadiene copolymer type.

As hydrophobized clays, mention may be made of disteardimonium hectorite such as Bentone 38 VCG, Bentone Gel ISD V or Bentone Gel IHD V sold by Elementis; mention may also be made of stearalkonium hectorite such as Bentone 27 V CG sold by Elementis. As hydrophobized silica, mention may be made of aerosil R972 sold by Wacker or else Evonik Degussa.

As silicone elastomer, mention may be made of dimethicone/vinyl dimethicone crosspolymer such as KSG-6 and KSG-16 sold by Shin-Etsu, or vinyl dimethicone/methicone silsesquioxane crosspolymer such as KSP 100 sold by Shin-Etsu.

As oil gelling polymer, mention may be made of ethylene diamine/stearyl dimer dilinoleate copolymer such as Oleocraft LP-10-PA-(MV) sold by Croda, polyamide-8 such as Oleocraft LP-20-PA-(MV) sold by Croda, poly C₁₀-C₃₀ alkyl acrylate such as Intelimer IPA 13-6 or Intelimer IPA 13-1 NG Polymer sold by Air Products & Chemicals, nylon-611/dimethicone copolymer such as Dow Corning 2-8179 Gellant sold by Dow Corning, or dextrin palmitate such as rheopearl KL2-OR sold by Chiba Flour Milling.

As block polymer of hydrogenated styrene/butadiene copolymer type, mention may be made of the Kratons G1701 EU SQR 1111 and G1657 MS 1111.

The fatty phase gelling agent may be present in the compositions of the invention at a content ranging from 0.1% to 15% by weight relative to the total weight of the composition, preferably from 0.5% to 12% by weight, and even more preferably from 1% to 10%.

According to a particularly preferred form of the invention, the compositions will be transparent and will preferably have a light transmittance, at a wavelength equal to 500 nm, through a sample 1 cm thick, of greater than 10% (which corresponds to an absorbance [abs=−log (transmittance)] of less than 1.5), preferably greater than 30%, better still than 50% and even more preferentially greater than 70%.

According to one particularly preferred form of the invention, the compositions will have an SPF of greater than or equal to 15, or even greater than or equal to 20 or even greater than or equal to 30.

According to one particularly preferred form of the invention, the compositions will have a UVA_{PPD} PF of greater than or equal to 5, and they also satisfy the regulation, especially the European regulation, which stipulates that the SPF/PPD ratio should be less than 3.

### Additives

The anhydrous composition of the invention may also contain various additives that may be soluble in the oily phase, or that may be in dispersion in said oily phase, chosen especially from lipophilic dyes, lipophilic active agents, lipophilic polymers, organic solvents, preserving agents, insect repellents, essential oils, fragrances, emollients, propellants, fatty substances, humectants and silicones.

Among the lipophilic cosmetic active agents, examples that may be mentioned include antioxidants, keratolytic agents such as N-alkylsalicylic acids, for example N-octanoyl-5-salicylic acid; vitamins such as vitamin E (tocopherol and derivatives), vitamin A (retinol and derivatives); softeners and any lipophilic active agent usually used for caring for the skin or the hair.

Additional lipophilic polymers that may be mentioned include styrene-based block copolymers such as styrene/ethylene-butylene/styrene copolymer such as the product sold under the name Kraton G-1650E by Kraton Polymers; acrylic or methacrylic acid copolymers, such as the acrylate/stearyl acrylate/dimethicone methacrylate copolymer sold under the name KP 561 P by the company Shin-Etsu; poly-C₁₀-C₃₀-alkyl acrylates, such as the product sold under the name Intelimer IPA 13-1 by Landec.

Of course, those skilled in the art will take care to choose the abovementioned optional additional compound or compounds and/or their amounts so that the advantageous properties intrinsically attached to the compositions in accordance with the invention are not, or not substantially, detrimentally affected by the envisaged addition or additions.

Another subject of the present invention consists of the use of the compositions according to the invention as defined above for the manufacture of products for the cosmetic treatment of the skin, the nails, the hair, the eyelashes, eyebrows and/or the scalp, especially care products and anti-sun products.

The cosmetic compositions according to the invention may be used, for example, as care products and/or anti-sun products and/or daily photoprotection products and/or makeup products and/or hair products, for the face and/or the body and/or the hair, preferably of a liquid consistency.

### Additional Colouring Agents

According to another particular form of the invention, the compositions of the invention may also comprise one or more additional colouring agents.

The additional colouring agents may also be chosen from natural or synthetic direct dyes. They may be organic or mineral dyes.

The natural or synthetic liposoluble organic dyes are, for example, DC Red 17, DC Red 21, DC Red 27, DC Green 6, DC Yellow 11, DC Violet 2, DC Orange 5, Sudan red, carotenes (β-carotene or lycopene), xanthophylls (capsanthin, capsorubin or lutein), palm oil, Sudan brown, quinoline yellow, annatto and curcumin.

The additional colouring agents may also be chosen from particulate colorants, which are preferably chosen from pigments, nacres or interference pigments, and glitter flakes.

The term “pigments” should be understood as meaning white or coloured, mineral or organic particles of any form, which are insoluble in the physiological medium and are intended to colour the composition.

The pigments may be white or coloured, and mineral and/or organic. Among the mineral pigments, mention may be made of titanium dioxide, optionally surface-treated, zirconium oxide or cerium oxide, and also zinc oxide, iron (black, yellow or red) oxide or chromium oxide, manganese violet, ultramarine blue, chromium hydrate and ferric blue, and metal powders, for instance aluminium powder and copper powder.

Among the organic pigments that may be mentioned are carbon black, pigments of D & C type and lakes based on cochineal carmine or on barium, strontium, calcium or aluminium.

Mention may also be made of pigments with an effect, such as particles comprising a natural or synthetic, organic or mineral substrate, for example glass, acrylic resins, polyester, polyurethane, polyethylene terephthalate, ceramics or aluminas, said substrate being uncoated or coated with metallic substances, for instance aluminium, gold, silver, platinum, copper or bronze, or with metal oxides, for instance titanium dioxide, iron oxide or chromium oxide, and mixtures thereof.

Within the meaning of the present invention, the term “interference particles or nacres” denotes any particle generally having a multilayer structure such that it allows the creation of a colour effect by interference of light rays, which diffract and scatter differently according to the nature of the layers. The colouring effects obtained are associated with the lamellar structure of these particles and are derived from the physical laws of thin film optics (see: Pearl Lustre Pigments—Physical principles, properties, applications—R. Maisch, M. Weigand. Verlag Moderne Industrie). Thus, these particles may have colours that vary according to the angle of observation and the incidence of the light.

Within the meaning of the present invention, a multilayer structure is intended to denote, without preference, a structure formed from a substrate coated with a single layer, or a structure formed from a substrate coated with at least two or even more consecutive layers.

The multilayer structure may thus comprise one or even at least two layers, each layer, independently or otherwise of the other layer(s), being made of at least one material chosen from the group consisting of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite and alloys, polymers and combinations thereof.

Generally, the multilayer structure is of mineral nature.

More particularly, the interference particles under consideration according to the invention may be interference pigments, or alternatively natural or synthetic, monolayer or multilayer nacres, in particular formed from a natural substrate based, inter alia, on mica, which is covered with one or more layers of metal oxide.

The interference particles according to the invention are characterized in that 50% of the mass population has a diameter (d50) of less than 40 μm, more particularly less than 30 μm, especially less than 20 μm and in particular less than 15 μm, measured with a laser granulometer, for instance the Mastersizer 2000® machine from Malvern or the BI90+® machine from Brookhaven Instrument Corporation.

Nacres of mica/tin oxide/titanium oxide type, for instance those sold under the names Timiron Silk Blue®, Timiron Silk Red®, Timiron Silk Green®, Timiron Silk Gold® and Timiron Super Silk® sold by Merck, and mica/iron oxide/titanium oxide nacres, for instance Flamenco Satin Blue®, Flamenco Satin Red® and Flamenco Satin Violet® and Flamenco Orange 320C sold by Engelhard, and mixtures thereof, are most particularly suitable for the invention.

More specifically, these pigments may be present in amounts ranging from 0.01% to 10% by weight and preferably ranging from 0.1% to 5% by weight relative to the total weight of the composition.

### Compositions

The compositions according to the invention are preferably vaporizable and/or sprayable by means of devices well known to those skilled in the art.

The compositions according to the invention may be in the form of a vaporizable oil applied to the skin or the hair in the form of fine particles by means of pressurization devices. The devices in accordance with the invention are well known to those skilled in the art and comprise non-aerosol pumps or “atomizers”, aerosol containers comprising a propellant and aerosol pumps using compressed air as propellant. The latter are for example described in U.S. Pat. No. 4,077,441 and U.S. Pat. No. 4,850,517 (forming an integral part of the content of the description).

These compositions may also be impregnated onto supports such as wipes, or they may be packaged as lotions in a bottle with a reducing agent.

The compositions packaged in aerosol form in accordance with the invention generally contain conventional propellants, for instance the hydrofluoro compounds dichlorodifluoromethane, difluoroethane, dimethyl ether, isobutane, n-butane, propane or trichlorofluoromethane. They are preferably present in amounts ranging from 15% to 70% by weight, relative to the total weight of the composition.

Concrete but in no way limiting examples illustrating the invention will now be given.

### EXAMPLES

### Examples 1 to 3

The anhydrous formulations were prepared: The amounts are expressed as weight % relative to the total weight of the composition.

| Phase | Ingredients | Ex. 1 | Ex. 2 | Ex. 3 |
|---|---|---|---|---|
| | DIISOPROPYL SEBACATE | 4.9 | 16.9 | 22.05 |
| | BUTYL | 5 | 5 | 5 |
| | METHOXYDIBENZOYLMETHANE | | | |
| | ETHYLHEXYL TRIAZONE | 2.5 | 2.5 | 2.5 |
| | OCTOCRYLENE | 5 | 5 | 5 |
| | ETHYLHEXYL SALICYLATE | 5 | 5 | 5 |
| | DROMETRIZOLE TRISILOXANE | 0.5 | 0.5 | 0.5 |
| | BIS-ETHYLHEXYLOXYPHENOL | 2.5 | 2.5 | 2.5 |
| | METHOXYPHENYL TRIAZINE | | | |
| | HOMOSALATE | 10 | 10 | 10 |
| | POLYAMIDE-8 (OLEOCRAFT LP- | 2.9 | 2.9 | 2.9 |
| | 20-PA-(MV) from CRODA) | | | |
| B | 96° ETHANOL | 12 | 0 | 12 |
| | PHENYL TRIMETHICONE | 2 | 2 | 2 |
| | CYCLOHEXASILOXANE | 18 | 18 | 13 |
| | DIMETHICONE (XIAMETER PMX- | 12.15 | 12.15 | 0 |
| | 200 SILICONE FLUID 5CS from | | | |
| | DOW CORNING) | | | |
| | PEG-10 DIMETHICONE (KF-6017 | 1.5 | 1.5 | 1.5 |
| | from SHIN-ETSU) | | | |
| | ISODODECANE | 16.05 | 16.05 | 16.05 |

The procedure for compositions 1 to 3 is as follows:

Phase A is prepared by mixing the starting materials with mechanical stirring at 80° C.; phase B is prepared by mixing the starting materials with mechanical stirring at room temperature; the solutions obtained are macroscopically homogeneous. Phase A is cooled, with slow stirring, to 40° C.; then phase B is added by slow introduction into phase A with stirring using a COS 1000 type homogenizer at a stirring speed of 4000 rpm for 15 minutes. The homogeneous solution obtained is cooled with stirring at room temperature. The solution obtained is homogeneous, fluid and transparent. The viscosity of the compositions 1, 2 and 3 at 25° C. is equal to 0.01 Pa·s (measured using a Rheomat 180 (from Lamy), equipped with an MS-R1, MS-R2, MS-R3, MS-R4 or MS-R5 spindle chosen according to the consistency of the composition, rotating at a rotation speed of 200 rpm).

The stability and shine following application of these 3 compositions were evaluated.

### Procedure for Evaluating Shine Following Application

The composition to be evaluated is applied to a contrast sheet at an amount of 6 mg/cm²: the formulation is deposited by means of a micropipette then spread very quickly over a circular surface area with a radius of 1 cm by a finger stall in order to distribute it very uniformly. The film obtained is dried for 24 hours at 25° C. (degree of humidity: 50% RH).

The shiny character of the film obtained is evaluated by means of a glossmeter of BYK Gardner, micro-gloss 60° type.

### Results

TABLE I

| | | Shine following |
|---|---|---|
| | Stability | application |
| Example 1 according | ++++ | Low: 72.4 |
| to the invention | | |
| Comparative | −−−− | Average: 85 |
| Example 2 | Opaque, | |
| | heterogeneous | |
| | formulation | |
| Comparative | ++++ | High: 92.7 |
| Example 3 | | |

These results show that only the composition according to the invention, comprising at least one lipophilic screening system at a content of greater than or equal to 5%, at least 20% of a silicone fatty phase and at least one monoalcohol, is stable and has excellent cosmetic properties, and especially a low shine effect following application.

Finally, the composition of the invention described in example 1 is stable, has good photoprotection properties, with an in vivo SPF level of approximately 42 and a persistence in water equal to 52.7%. The composition of example 1 moreover has excellent cosmetic properties following application, with no whitening effect on the skin and no tacky feel.

These oils are stable after storage for 2 months at different temperatures (4, 25, 45° C.). They have the advantage of being vaporizable in the form of a very fine mist without aerosol.

### Transparency Measurement:

The transmittance of the compositions of examples 1 to 3 is equal to approximately 95%, according to the protocol described previously.

### Examples 4 and 5

The following compositions were prepared:

| | COMPO- | COMPO- |
|---|---|---|
| INCI (EU) | SITION 4 | SITION 5 |
| DISTEARDIMONIUM | 0.8 | 0.8 |
| HECTORITE | | |
| SILICA SILYLATE | 0.5 | 0.5 |
| CI 77891 (and) DISODIUM | | 0.9 |
| STEAROYL GLUTAMATE (and) | | |
| ALUMINUM HYDROXIDE | | |
| CI 77491 (and) DISODIUM | | 0.144 |
| STEAROYL GLUTAMATE (and) | | |
| ALUMINUM HYDROXIDE | | |
| CI 77492 (and) DISODIUM | | 0.279 |
| STEAROYL GLUTAMATE (and) | | |
| ALUMINUM HYDROXIDE | | |
| SYNTHETIC | | 0.147 |
| FLUORPHLOGOPITE | | |
| DIISOPROPYL SEBACATE | 4.9 | 4.9 |
| BUTYL | 5 | 5 |
| METHOXYDIBENZOYLMETHANE | | |
| ETHYLHEXYL SALICYLATE | 5 | 5 |
| ETHYLHEXYL TRIAZONE | 2.5 | 2.5 |
| OCTOCRYLENE | 5 | 5 |
| HOMOSALATE | 10 | 10 |
| DROMETRIZOLE TRISILOXANE | 2.5 | 0.5 |
| BIS-ETHYLHEXYLOXYPHENOL | | 2 |
| METHOXYPHENYL TRIAZINE | | |
| MICA (and) CI 77891 | | 3 |
| ETHYLENEDIAMINE/STEARYL | 2.75 | 2 |
| DIMER DILINOLEATE | | |
| COPOLYMER | | |
| PHENYLTRIMETHICONE | 2 | 2 |
| CYCLOHEXASILOXANE | 18 | 18 |
| DIMETHICONE | 12.5 | 7.74 |
| VINYL | 4.8 | 4.8 |
| DIMETHICONE/METHICONE | | |
| SILSESQUIOXANE | | |
| CROSSPOLYMER (KSP 100 from | | |
| SHIN-ETSU) | | |
| PEG-10 DIMETHICONE (KF-6017 | 1.5 | 1.5 |
| from SHIN-ETSU) | | |
| 96° ETHANOL | 6 | 6 |
| PROPYLENE CARBONATE | 0.2 | 0.2 |
| ISODODECANE | 16.05 | 17.09 |

Composition 4 is in the form of an opalescent serum with a high SPF (in vivo SPF of 50) giving a non-tacky film following application to the skin.

Composition 5 is in the form of a CC product with a “shaka shaka” texture (fluid texture known in Asia, which is applied following shaking in order to ensure the homogeneity of the composition) with a high SPF (in vivo SPF of 50) giving a non-tacky film following application to the skin, with skin tone correcting properties.

### Example 6

The following composition was prepared:

| INCI (EU) | COMPOSITION 6 |
|---|---|
| DISTEARDIMONIUM HECTORITE | 0.8 |
| DISTEARDIMONIUM HECTORITE | 10 |
| (and) PROPYLENE CARBONATE | |
| SILICA SILYLATE | 0.5 |
| DIISOPROPYL SEBACATE | 4.9 |
| BUTYL | 5 |
| METHOXYDIBENZOYLMETHANE | |
| ETHYLHEXYL SALICYLATE | 5 |
| ETHYLHEXYL TRIAZONE | 1.5 |
| OCTOCRYLENE | 5 |
| HOMOSALATE | 10 |
| DROMETRIZOLE TRISILOXANE | 0.5 |
| BIS-ETHYLHEXYLOXYPHENOL | 2 |
| METHOXYPHENYL TRIAZINE | |
| ETHYLENEDIAMINE/STEARYL | 2 |
| DIMER DILINOLEATE COPOLYMER | |
| PHENYL TRIMETHICONE | 2 |
| CYCLOHEXASILOXANE | 14.1 |
| DIMETHICONE (and) | 13 |
| DIMETHICONE/VINYL | |
| DIMETHICONE CROSSPOLYMER | |
| (75/25) (KSG 16 from SHIN-ETSU) | |
| PEG-10 DIMETHICONE (KF-6017 | 1.5 |
| from SHIN-ETSU) | |
| 96° ETHANOL | 6 |
| PROPYLENE CARBONATE | 0.2 |
| ISODODECANE | 16 |

Composition 6 is in the form of an opalescent gel with a viscosity, measured at 25° C., equal to 0.366 Pa·s (measured using a Rheomat 180 (from Lamy), equipped with an MS-R1, MS-R2, MS-R3, MS-R4 or MS-R5 spindle chosen according to the consistency of the composition, rotating at a rotation speed of 200 rpm).

When applied to the skin, the composition is non-tacky and non-shiny.

1. An anhydrous composition comprising:
a) at least 20% by weight, relative to the total weight of the composition, of a silicone fatty phase,
b) at least 5% by weight, relative to the total weight of the composition, of one or more lipophilic organic UV screening agents, and
c) at least one or more C₂-C₈ monoalcohols.

2. The composition according to claim 1, wherein the silicone fatty phase is present in an amount of greater than 22% by weight relative to the total weight of the composition.

3. The composition according to claim 1, wherein the silicone fatty phase comprises one or more volatile or non-volatile silicone oils.

4. The composition according to claim 1, wherein the silicone fatty phase comprises one or more volatile silicone oils chosen from octamethylcyclotetrasiloxane, decamethyl cyclopentasiloxane, dodecamethyl cyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

5. The composition according to claim 1, wherein the silicone fatty phase comprises one or more non-volatile silicone oils chosen from non-volatile polydimethylsiloxanes (PDMS s), polydimethylsiloxanes comprising alkyl or alkoxy groups that are pendent and/or at the end of a silicone chain, these groups each having from 2 to 24 carbon atoms, phenyl silicones and mixtures thereof.

6. The composition according to claim 1, wherein the lipophilic organic screening agent(s) are chosen from para-aminobenzoic acid derivatives, salicylic derivatives, cinnamic derivatives, benzophenones and aminobenzophenones, anthranilic derivatives, dibenzoylmethane derivatives, β,β-diphenylacrylate derivatives, benzylidenecamphor derivatives, phenylbenzimidazole derivatives, benzotriazole derivatives, triazine derivatives, bis-resorcinyl triazines, imidazoline derivatives, benzalmalonate derivatives, 4,4-diarylbutadiene derivatives, benzoxazole derivatives and merocyanines, and mixtures thereof.

7. The composition according to claim 1, wherein the lipophilic organic screening agent(s) are present in the compositions according to the invention in proportions ranging from 5% to 50% by weight relative to the total weight of the composition.

8. The composition according to claim 1, wherein the C₂-C₈ monoalcohol(s) are chosen from ethanol, propanol, isopropanol and n-butanol.

9. Composition according to claim 1, wherein the amount of monoalcohol(s) ranges from 1% to 30% by weight relative to the total weight of said composition.

10. The composition according to claim 1, which is transparent.

11. The composition according to claim 1, which has an SPF of greater than or equal to 15.

12. The composition according to claim 1, which has a UVA_{PPD} PF of greater than 5 and/or an SPF/PPD ratio of less than 3.

13. The composition according to claim 1, which also comprises one or more cosmetic adjuvants chosen from lipophilic dyes, lipophilic active agents, lipophilic polymers, organic solvents, preserving agents, insect repellents, essential oils, fragrances, emollients, propellants, fatty substances, humectants and silicones.

14. The composition according to claim 1, which also comprises one or more silicone elastomers.

15. A process for the cosmetic treatment of the skin, the nails, the hair, the eyelashes, eyebrows and/or the scalp which comprises apply, to the surface of the skin, the nails, the hair, the eyelashes, eyebrows and/or the scalp at least one composition as defined in claim 1.

16. A cosmetic process for limiting the darkening of the skin and/or improving the colour and/or uniformity of the complexion, comprising the application, to the surface of the skin, of at least one composition as defined in claim 1.

17. A cosmetic process for preventing and/or treating the signs of ageing of a keratin material, comprising the application, to the surface of the keratin material, of a composition as defined in claim 1.

18. The composition according to claim 2, wherein the silicone fatty phase comprises one or more volatile or non-volatile silicone oils.

19. The composition according to claim 2, wherein the silicone fatty phase comprises one or more volatile silicone oils chosen from octamethylcyclotetrasiloxane, decamethyl cyclopentasiloxane, dodecamethyl cyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

20. The composition according to claim 3, wherein the silicone fatty phase comprises one or more volatile silicone oils chosen from octamethylcyclotetrasiloxane, decamethyl cyclopentasiloxane, dodecamethyl cyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.
